(19) United States
(12) Patent Application Publication
(10) Pub. No.: US 2026/0198504 A1
(43) Pub. Date: Jul. 16, 2026

(54) COMPOSITION OF AN ALGAL EXTRACT

(71) Applicants: UPL MAURITIUS LIMITED, Port Louis (MU); UPL EUROPE SUPPLY CHAIN GMBH, Rotkreuz/Risch (CH)

(72) Inventors: Gregory LECOLLINET, Saint-Malo (FR); Sheldon PARK, Guelph (CA); Lisa PARK, Guelph (CA)

(73) Assignees: UPL MAURITIUS LIMITED, Port Louis (MU); UPL EUROPE SUPPLY CHAIN GMBH, Rotkreuz/Risch (CH)

(21) Appl. No.: 19/138,792

(22) PCT Filed: Dec. 21, 2023
(86) PCT No.: PCT/IB2023/063099

§ 371 Date: Jun. 13, 2025

(30) Foreign Application Priority Data
Dec. 23, 2022 (EP) 22307039.2

(51) Int. Cl. A01N 65/03; A01N 25/22; A01P 21/00; G01N 33/84

(52) U.S. Cl. CPC A01N 65/03; A01N 25/22; A01P 21/00; G01N 33/84; G01N 2333/405

(57) ABSTRACT

The present disclosure relates to a composition comprising an algal extract. More specifically, the present disclosure relates to an agricultural composition comprising a concentrated algal extract, preservative and optionally, a diluent. The present disclosure further relates to a method of producing the algal extract and uses of the composition described herein.

### FIELD OF INVENTION

The present disclosure relates to a composition comprising an algal extract. More specifically, the present disclosure relates to an agricultural composition comprising a concentrated algal extract, preservative and optionally, a diluent. The present disclosure further relates to a method of producing the algal extract and uses of the composition described herein.

### BACKGROUND

Increasing yield has been a major goal for the farming community given the rise in demand for food and plant-based products. Traditionally, NPK fertilizers have been used by growers to achieve the goal. Over a period, several problems associated with synthetic and chemical fertilizers have arisen such as run-offs, leaching, soil degradation, soil microbiome reduction, and so forth. As a result, there is a demand for sustainable practices such as use of biofertilizers, soil conditioners and biostimulants, particularly the ones that are derived from natural sources.

Algae play an important role in agriculture where they are used as biofertilizer and soil stabilizers. Fertilizers and plant growth regulators (PGRs) based on algae, such as seaweeds (marine algae) are commonly known, having been produced and utilized for dozens of years. Algal based fertilizers and soil conditioners contain algal extracts, for example seaweed extracts, which are rich in bioactive ingredients, phytostimulants, minerals, proteins, and other compounds beneficial for plant growth. It is very important to have robust processes of producing such extracts so that the functional characteristics of the extracts are preserved, if not enhanced. Also, being a product of natural and biological origin, stability, storage, logistics and functional viability remains a challenge for algal extracts and products comprising them.

EP0538091 teaches a composition derived from marine algae obtained by a process comprising the following steps:
a) basic hydrolysis of at least one brown alga in the presence of a reducing agent, at a temperature between 5° and 100° C., for a period of between 4 and 8 h;
b) neutralization of the hydrolysate obtained in this way with a strong acid, until a pH of between 6 and 8 is obtained;
c) filtration;
d) optionally diafiltration or electrodialysis of the permeate obtained at the end of step c); and
e) optionally reducing, to the powder thereof, the permeate obtained at the end of step c) or the retentate obtained at the end of step d).

Nonetheless, due to the basic hydrolysis of the alga under hot conditions in the presence of a reducing agent, some compounds of the intracellular fluid of the alga are not preserved, especially polysaccharides, lipids, proteins and polyphenols, which reduces the biostimulating properties of the composition obtained with the process described above.

FR3025699 teaches a concentrated algae extract characterized in that it has a percentage of dry matter between 6 and 100% and a process for preparation the extract. The process comprises following steps:
crush the algae until cell burst;
homogenize the ground algae in aqueous solution;
remove alginate and cellulose by flocculation and filtration;
possibly adjust the pH of the filtrate;
concentrate the filtrate to reach a percentage of dry matter of between 6 and 100%, preferably between 9 and 70%, more preferably between 12 and 50%.

WO2021069866A1 teaches a seaweed algal extract comprising dry matter between about 18%-36% and process for making the extract. The process comprises following steps: a. feeding an initial algal extract comprising dry matter between 3-8% to an evaporator heat exchanger; and b. passing steam through the heat exchanger to produce the algal extract having dry matter between about 18%-36%.

WO2018075943 teaches a method of modulating plant growth comprising administering to a plant an amount of a composition comprising an amount of an Ascophyllum composition that is effective to detectably modulate at least one aspect of growth in the plant, at least one health characteristic in the plant, or both. The aspects may be selected from increased root growth, increased total plant biomass, improved resistance to salt stress, improved resistance to temperature stress, and improved resistance to chlorosis. The disclosure states that “In some embodiments, the macroalgae composition, such as the Ascophyllum material (e.g., the Ascophyllum extract(s) that form part of or all of the active ingredient portion of the composition), comprises 50% or less by volume of the entire composition. In some embodiments, the active ingredient components or extracts may comprise 40% or less by volume of the composition. In some embodiments, the active ingredient components or extracts may comprise 30% or less by volume of the composition. In some embodiments, the active ingredient components or extracts may comprise 20% or less by volume of the composition.”

However, there is continuous need in the domain to have a composition comprising an algal extract that is concentrated, effective, stable and easy to use. Further, another issue that lies with liquid and concentrated algal extract compositions is their degradation and shorter shelf life due to the presence of large amount of organic content.

Despite the knowledge panning years about the algal-based products and their application, there continues to be a need for additional ways of modulating the growth and/or health of plants. In quest of this need, it is essential to use an algal-based product which is stable, rich in its bioactive content, and is functionally viable. It is also a necessity to have robust and optimized processes and biomarkers concentration to characterize the quality of the concentrated extract. However, prior art methods of preparing, characterizing, and using compositions derived from algae, such as seaweeds, are often ineffective or limited in their applicability with respect to their stability, concentrations and in affecting plant growth and/or health characteristics. Moreover, there still remains a need for algal extract-based compositions which can be applied in low doses to generate higher yield, growth and stress tolerance in plants. It is dire need in the domain to have composition with concentrated algal extract that are efficacious and at the same time have longer shelf life.

The present disclosure aims to provide compositions and methods that are markedly different from such compositions known in the prior art and provide new and effective ways of modulating plant health and/or growth characteristics. The present disclosure aims to provide a composition comprising an algal extract, wherein the composition is stable, concentrated with bioactives and nutrients, and is functionally viable. The present disclosure aims to provide a process of making concentrated algal extract which is stable, and functionally effective in affecting yield, plant growth and/or health characteristics. The inventors of the present disclosure herein aim to provide an algal-based agricultural composition which benefits the plant in terms of yield, growth and stress tolerance, even at lower doses. The present disclosure also aims to provide a way to characterize the algal extracts by quantitating the biomarkers.

### SUMMARY

An aspect of the present disclosure provides a composition comprising a concentrated algal extract, a preservative and optionally, a diluent; wherein the concentrated algal extract is present in an amount ranging from about 50% w/w to about 99.75% w/w of the total weight of the composition, the concentration of the preservative is 0.15 w/w to 50% w/w of the total weight of the composition and the diluent is present in an in an amount ranging from about 0% w/w to about 50% w/w of the total weight of the composition.

An aspect of the present disclosure also provides methods of preparation of the compositions described herein.

In an aspect, the concentrated algal extract is characterized by presence of any one or more of biomarkers selected from the group consisting of
a) Mannitol present in an amount ranging from about 1.5% w/w to about 7% w/w of the total weight of the extract;
b) Fucoidans present in an amount ranging from about 0.4% w/w to about 1.5% w/w of the total weight of the extract;
c) Polyphenols present in an amount ranging from about 0.2% w/w to about 5% w/w of the total weight of the extract;
d) Glycine betaine present in an amount ranging from about 0.0001% w/w to about 0.005% w/w of the total weight of the extract; and
e) Laminine present in an amount ranging from about 0.01% w/w to about 0.05% w/w of the total weight of the extract.

In an aspect, the concentrated algal extract is an acidic extract with a pH ranging between 2 and 4, wherein the extract is acidified using an acid; wherein acid is selected from a weak acid, mild acid and strong acid; more preferably the acid is a weak acid or mild acid.

In an aspect of the invention, the weak or mild acid is selected from organic acids, oxalic acid, sulfurous acid, phosphoric acid, hydrogen sulfurous ion, nitrous acid, hydrofluoric acid, methanoic acid, benzoic acid, acetic acid, formic acid, citric acid, acetic acid, tartaric acid, ascorbic acid, lactic acid, uric acid, malic acid, succinic acid, propionic acid, acrylic acid, glyceric acid, carbonic acid, glycolic acid, glyoxylic acid, water, conjugates of weak bases, hydrocyanic and combinations thereof. In an aspect of the invention, the organic acid is characterized by a carboxyl (—COOH) functional group, wherein the number of carbon atoms in the organic acid is C₁ to C₂₆.

In an aspect, the concentrated algal extract is prepared by a process comprising
i) crushing the algal cells to cause cell lysis;
ii) homogenizing the lysed algal cells in an aqueous solution;
iii) adding an acid to the homogenized lysed algal cells in an aqueous solution;
iv) removing alginate and cellulose by acidic flocculation and filtration; and
v) concentrating the filtrate to obtain the concentrated algal extract having a pH ranging between 2 and 4.

In an embodiment, the concentrated algal extract is an extract of seaweeds. In an embodiment, the concentrated algal extract is an extract of brown algae. In an embodiment, the concentrated algal extract is an extract of algae selected from Ascophyllum, Fucus, Laminaria, Ecklonia, Sargassum, Lemna, Kappaphycus, Gracilaria, Macrocystis, Turbinaria, Durvilea, and their mixtures. In an embodiment, the concentrated algal extract is an extract of algae selected from Ascophyllum nodosum, Fucus vesiculosus, Laminaria digitata, Laminaria hyperborea, Laminaria saccharina, Ecklonia maxima, Sargassum spp., and their mixtures. In a preferred embodiment, the concentrated algal extract is an extract of Ascophyllum nodosum.

In an aspect, the present disclosure provides a method of growth stimulation in a target plant, wherein the method comprises applying the compositions described herein to a target plant, a locus or a seed.

An aspect of the present disclosure also provides a method of determining the quality of the composition of the present disclosure, said method comprising
i) obtaining a sample of said composition; and
ii) measuring the pH and the concentration of any one or more of biomarkers selected from mannitol, glycine betaine, fucoidans, polyphenols and laminine in the sample of said composition;
   wherein said composition is considered stable and good provided the pH of the sample of said composition is detected to be in a range of 2 to 4 and wherein the concentration of any one or more of biomarkers selected from mannitol, glycine betaine, fucoidans, polyphenols and laminine are detected to be in a range as provided in Table 2.

### DETAILED DESCRIPTION

For the purposes of the following detailed description, it is to be understood that the invention may assume various alternative variations and step sequences, except where expressly specified to the contrary. Moreover, other than in any operating examples, or where otherwise indicated, all numbers expressing, for example, quantities of materials/ingredients used in the specification are to be understood as being modified in all instances by the term “about”.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate). As used herein, the term “about” refers to a measurable value such as a parameter, an amount, a temporal duration, and the like and is meant to include variations of +/−15% or less, specifically variations of +/−10% or less, more specifically variations of +/−5% or less, even more specifically variations of +/−1% or less, and still more specifically variations of +/−0.1% or less of and from the particularly recited value, in so far as such variations are appropriate to perform in the disclosure described herein.

Recitation of ranges of values are merely intended to serve as a shorthand method of referring individually to each separate value falling within the range, unless otherwise indicated herein, and each separate value is incorporated into the specification as if it were individually recited herein. All provided ranges of values are intended to include the end points of the ranges, as well as values between the end points, and are independently combinable. As used herein, all numerical values or numerical ranges include integers within such ranges and fractions of the values or the integers within ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. All methods described herein can be performed in a suitable order unless otherwise indicated herein or otherwise clearly contradicted by context.

It must be noted that, as used in this specification, the singular forms “a,” “an” and “the” include singular and plural referents unless the content clearly dictates otherwise. The terms “preferred” and “preferably” refer to embodiments of the invention that may afford certain benefits, under certain circumstances.

As used herein, the terms “comprising” “including,” “having,” “containing,” “involving,” and the like are to be understood to be open-ended, i.e., to mean including but not limited to.

Thus, before describing the present disclosure in detail, it is to be understood that this invention is not limited to particularly exemplified process parameters or composition that may of course, vary. It is also to be understood that the terminology used herein is for the purpose of describing embodiments of the invention only and is not intended to limit the scope of the invention in any manner. The use of examples anywhere in this specification including examples of any terms discussed herein is illustrative only, and in no way limits the scope and meaning of the invention or of any exemplified term. Likewise, the invention is not limited to various embodiments given in this specification. Unless otherwise defined, all technical and scientific terms used herein have the same meaning as commonly understood by one of ordinary skill in the art to which this invention pertains. In the case of conflict, the present document, including definitions will control.

An embodiment of the present disclosure provides a composition comprising a concentrated algal extract, a preservative and optionally, a diluent; wherein the concentrated algal extract is present in an amount ranging from about 50% w/w to about 99.75% w/w of the total weight of the composition, the concentration of the preservative is 0.15 w/w to 50% w/w of the total weight of the composition and the diluent is present in an in an amount ranging from about 0% w/w to about 50% w/w of the total weight of the composition.

An embodiment of the present disclosure provides a composition comprising a concentrated algal extract, a preservative and optionally, a diluent; wherein the concentrated algal extract is present in an amount ranging from about 60% w/w to about 99.75% w/w of the total weight of the composition, the concentration of the preservative is 0.15 w/w to 50% w/w of the total weight of the composition and the diluent is present in an in an amount ranging from about 0% w/w to about 50% w/w of the total weight of the composition.

An embodiment of the present disclosure provides a composition comprising a concentrated algal extract, a preservative and optionally, a diluent; wherein the concentrated algal extract is present in an amount ranging from about 70% w/w to about 99.75% w/w of the total weight of the composition, the concentration of the preservative is 0.15 w/w to 50% w/w of the total weight of the composition and the diluent is present in an in an amount ranging from about 0% w/w to about 50% w/w of the total weight of the composition.

An embodiment of the present disclosure provides a composition comprising a concentrated algal extract, a preservative and optionally, a diluent; wherein the concentrated algal extract is present in an amount ranging from about 80% w/w to about 99.75% w/w of the total weight of the composition, the concentration of the preservative is 0.15 w/w to 50% w/w of the total weight of the composition and the diluent is present in an in an amount ranging from about 0% w/w to about 50% w/w of the total weight of the composition.

An embodiment of the present disclosure provides a composition comprising a concentrated algal extract, a preservative and optionally, a diluent; wherein the concentrated algal extract is present in an amount ranging from about 90% w/w to about 99.75% w/w of the total weight of the composition, the concentration of the preservative is 0.15 w/w to 10% w/w of the total weight of the composition and the diluent is present in an in an amount ranging from about 0% w/w to about 10% w/w of the total weight of the composition.

In a preferred embodiment, the concentrated algal extract is present in an amount ranging from about 90% w/w to about 99.75% w/w. In an embodiment, the concentrated algal extract is present in an amount ranging from about 91% w/w to about 99.75% w/w of the total weight of the composition. In an embodiment, the concentrated algal extract is present in an amount ranging from about 92% w/w to about 99.75% w/w of the total weight of the composition. In an embodiment, the concentrated algal extract is present in an amount ranging from about 93% w/w to about 99.75% w/w of the total weight of the composition. In an embodiment, the concentrated algal extract is present in an amount ranging from about 94% w/w to about 99.75% w/w of the total weight of the composition. In an embodiment, the concentrated algal extract is present in an amount ranging from about 95% w/w to about 99.75% w/w of the total weight of the composition. In an embodiment, the concentrated algal extract is present in an amount ranging from about 96% w/w to about 99.75% w/w of the total weight of the composition. In an embodiment, the concentrated algal extract is present in an amount ranging from about 97% w/w to about 99.75% w/w of the total weight of the composition. In an embodiment, the concentrated algal extract is present in an amount ranging from about 98% w/w to about 99.75% w/w of the total weight of the composition. In an embodiment, the concentrated algal extract is present in an amount ranging from about 99% w/w to about 99.75% w/w of the total weight of the composition. In a preferred embodiment, the concentrated algal extract is present in an amount ranging from about 99.2% w/w to about 99.75% w/w of the total weight of the composition. In another preferred embodiment, the concentration of the concentrated algal extract in the composition is 99.52% w/w.

In an embodiment, the concentrated algal extract is characterized by presence of any one or more of biomarkers selected from the group consisting of
a) Mannitol present in an amount ranging from about 1.5% w/w to about 7% w/w of the total weight of the extract;
b) Fucoidans present in an amount ranging from about 0.4% w/w to about 1.5% w/w of the total weight of the extract;
c) Polyphenols present in an amount ranging from about 0.2% w/w to about 5% w/w of the total weight of the extract;
d) Glycine betaine present in an amount ranging from about 0.0001% w/w to about 0.005% w/w of the total weight of the extract; and
e) Laminine present in an amount ranging from about 0.01% w/w to about 0.05% w/w of the total weight of the extract.

In an embodiment, the concentrated algal extract is an acidic extract with a pH ranging between 2 and 4.

In an embodiment, the concentrated algal extract is an extract of seaweeds. In an embodiment, the concentrated algal extract is an extract of brown algae. In an embodiment, the concentrated algal extract is an extract of algae selected from Ascophyllum, Fucus, Laminaria, Ecklonia, Sargassum, Lemna, Kappaphycus, Gracilaria, Macrocystis, Turbinaria, Durvilea, and their mixtures. In an embodiment, the concentrated algal extract is an extract of algae selected from Ascophyllum nodosum, Fucus vesiculosus, Laminaria digitata, Laminaria hyperborea, Laminaria saccharina, Ecklonia maxima, Sargassum spp., and their mixtures. In a preferred embodiment, the concentrated algal extract is an extract of Ascophyllum nodosum.

In an embodiment, the preservative is present in an in an amount ranging from about 0.15% w/w to about 50% w/w of the total weight of the composition. In an embodiment, the preservative is present in an in an amount ranging from about 0.15% w/w to about 40% w/w of the total weight of the composition. In an embodiment, the preservative is present in an in an amount ranging from about 0.15% w/w to about 30% w/w of the total weight of the composition. In an embodiment, the preservative is present in an in an amount ranging from about 0.15% w/w to about 20% w/w of the total weight of the composition. In an embodiment, the preservative is present in an in an amount ranging from about 0.15% w/w to about 10% w/w of the total weight of the composition. In an embodiment, the preservative is present in an amount ranging from about 0.15% w/w to about 5% w/w. In an embodiment, the preservative is present in an amount ranging from about 0.15% w/w to about 2% w/w. In an embodiment, the preservative is present in an amount ranging from about 0.15% w/w to about 1% w/w. In an embodiment, the preservative is present in an amount ranging from about 0.15% w/w to about 0.5% w/w. In an embodiment, the preservative is present in an amount ranging from about 0.15% w/w to about 0.3% w/w. In an embodiment, the preservative is present in an amount ranging from about 0.15% w/w to about 0.25% w/w. In a preferred embodiment, the preservative is present in an amount ranging from about 0.2% w/w to about 0.25% w/w.

In an embodiment, the preservative is selected from 1,2-benzisothiazolin-3-one; 2-Methyl-2H-isothiazol-3-one; sodium benzoate; potassium benzoate; benzoic acid; sorbic acid; potassium sorbate; sodium sulphite; potassium sulphite; sodium bisulphite; potassium bisulphite; sodium metabisulphite; potassium metabisulphite; sodium methylparaben; citric acid or salts thereof; malic acid or salts thereof; tartaric acid or salts thereof; propionic acid or salts thereof; acetic acid or salts thereof, and combinations thereof. In a preferred embodiment, the preservative is potassium sorbate. In a preferred embodiment, the preservative is sodium benzoate. In a preferred embodiment, the preservative is sodium methylparaben. In a preferred embodiment, the preservative is sodium metabisulphite.

In an embodiment, the composition of the present disclosure preferably comprises at least one preservative. Preferably, the preservative is potassium sorbate. In an embodiment, potassium sorbate is present in an in an amount ranging from about 0.15% w/w to about 50% w/w of the total weight of the composition. In an embodiment, potassium sorbate is present in an in an amount ranging from about 0.15% w/w to about 40% w/w of the total weight of the composition. In an embodiment, potassium sorbate is present in an in an amount ranging from about 0.15% w/w to about 30% w/w of the total weight of the composition. In an embodiment, potassium sorbate is present in an in an amount ranging from about 0.15% w/w to about 20% w/w of the total weight of the composition. In an embodiment, potassium sorbate is present in an in an amount ranging from about 0.15% w/w to about 10% w/w of the total weight of the composition. In a preferred embodiment, potassium sorbate is present in an amount ranging from about from about 0.15% w/w to about 0.5% w/w, more preferably 0.2% w/w to about 0.25% w/w of the total weight of the composition.

In an embodiment, the composition of the present disclosure preferably comprises at least one preservative. Preferably, the preservative is sodium benzoate. In an embodiment, sodium benzoate is present in an in an amount ranging from about 0.15% w/w to about 50% w/w of the total weight of the composition. In an embodiment, sodium benzoate is present in an in an amount ranging from about 0.15% w/w to about 40% w/w of the total weight of the composition. In an embodiment, sodium benzoate is present in an in an amount ranging from about 0.15% w/w to about 30% w/w of the total weight of the composition. In an embodiment, sodium benzoate is present in an in an amount ranging from about 0.15% w/w to about 20% w/w of the total weight of the composition. In a preferred embodiment, sodium benzoate is present in an amount ranging from about 0.15% w/w to about 0.5% w/w, more preferably from about 0.2% w/w to about 0.25% w/w of the total weight of the composition.

In an embodiment, the composition of the present disclosure preferably comprises at least one preservative. Preferably, the preservative is sodium methylparaben. In an embodiment, sodium methylparaben is present in an in an amount ranging from about 0.15% w/w to about 50% w/w of the total weight of the composition. In an embodiment, sodium methylparaben is present in an in an amount ranging from about 0.15% w/w to about 40% w/w of the total weight of the composition. In an embodiment, sodium methylparaben is present in an in an amount ranging from about 0.15% w/w to about 30% w/w of the total weight of the composition. In an embodiment, sodium methylparaben is present in an in an amount ranging from about 0.15% w/w to about 20% w/w of the total weight of the composition. In an embodiment, sodium methylparaben is present in an in an amount ranging from about 0.15% w/w to about 10% w/w of the total weight of the composition. In a preferred embodiment, sodium methylparaben is present in an amount ranging from about 0.15% w/w to about 0.5% w/w, more preferably from about 0.2% w/w to about 0.25% w/w of the total weight of the composition.

In an embodiment, the composition of the present disclosure preferably comprises at least two preservatives. The at least two preservatives may be selected from the preservatives disclosed in any of the above embodiments.

In a preferred embodiment, one of the preservative of the at least two preservatives is potassium sorbate. In another preferred embodiment, one of the preservative of the at least two preservatives is sodium metabisulphite.

In another preferred embodiment, the at least two preservatives are potassium sorbate and sodium metabisulphite. In an embodiment, potassium sorbate is present in an in an amount ranging from about 0.15% w/w to about 50% w/w of the total weight of the composition. In an embodiment, potassium sorbate is present in an in an amount ranging from about 0.15% w/w to about 40% w/w of the total weight of the composition. In an embodiment, potassium sorbate is present in an in an amount ranging from about 0.15% w/w to about 30% w/w of the total weight of the composition. In an embodiment, potassium sorbate is present in an in an amount ranging from about 0.15% w/w to about 20% w/w of the total weight of the composition. In an embodiment, potassium sorbate is present in an in an amount ranging from about 0.15% w/w to about 10% w/w of the total weight of the composition. In a preferred embodiment, potassium sorbate is present in an amount ranging from about from about 0.15% w/w to about 0.5% w/w, more preferably 0.2% w/w to about 0.25% w/w of the total weight of the composition. In an embodiment, sodium metabisulphite is present in an in an amount ranging from about 0.15% w/w to about 50% w/w of the total weight of the composition. In an embodiment, sodium metabisulphite is present in an in an amount ranging from about 0.15% w/w to about 40% w/w of the total weight of the composition. In an embodiment, sodium metabisulphite is present in an in an amount ranging from about 0.15% w/w to about 30% w/w of the total weight of the composition. In an embodiment, sodium metabisulphite is present in an in an amount ranging from about 0.15% w/w to about 20% w/w of the total weight of the composition. In an embodiment, sodium metabisulphite is present in an in an amount ranging from about 0.15% w/w to about 10% w/w of the total weight of the composition. In a preferred embodiment, sodium metabisulphite is present in an amount ranging from about from about 0.15% w/w to about 0.5% w/w, more preferably 0.2% w/w to about 0.25% w/w of the total weight of the composition.

In an embodiment, the compositions of the present disclosure optionally comprise a diluent. In an embodiment, the diluent may be present in an in an amount ranging from about 0% w/w to about 50% w/w of the total weight of the composition. In an embodiment, the diluent may be present in an in an amount ranging from about 0% w/w to about 40% w/w of the total weight of the composition. In an embodiment, the diluent may be present in an in an amount ranging from about 0% w/w to about 30% w/w of the total weight of the composition. In an embodiment, the diluent may be present in an in an amount ranging from about 0% w/w to about 20% w/w of the total weight of the composition. In an embodiment, the diluent may be present in an in an amount ranging from about 0% w/w to about 10% w/w of the total weight of the composition. In an embodiment, the diluent may be present in an amount ranging from about 0% w/w to about 5% w/w. In an embodiment, the diluent may be present in an amount ranging from about 0% w/w to about 2% w/w. In an embodiment, the diluent may be present in an amount ranging from about 0% w/w to about 1% w/w. In an embodiment, the diluent may be present in an amount ranging from about 0% w/w to about 0.5% w/w. In an embodiment, the diluent may be present in an amount ranging from about 0% w/w to about 0.3% w/w. In an embodiment, the diluent may be present in an amount ranging from about 0% w/w to about 0.3% w/w.

In an embodiment, the diluent may be a polar diluent. In an embodiment, the polar diluents are selected from water, acetone, acetonitrile, dimethylformamide (DMF), dimelthylsulfoxide (DMSO), isopropanol, and methanol. In an embodiment the polar diluent is water. Thus, in an embodiment, the composition of the present disclosure is an aqueous composition. In an embodiment, the polar diluent may be present in an in an amount ranging from about 0% w/w to about 50% w/w of the total weight of the composition. In an embodiment, the polar diluent may be present in an in an amount ranging from about 0% w/w to about 40% w/w of the total weight of the composition. In an embodiment, the polar diluent may be present in an in an amount ranging from about 0% w/w to about 30% w/w of the total weight of the composition. In an embodiment, the polar diluent may be present in an in an amount ranging from about 0% w/w to about 20% w/w of the total weight of the composition. In an embodiment, the polar diluent may be present in an in an amount ranging from about 0% w/w to about 10% w/w of the total weight of the composition. In a preferred embodiment, the polar diluent may be present in an amount ranging from about 0% w/w to about 0.5% w/w. In another preferred embodiment, the polar diluent may be present in an amount ranging from about 0% w/w to about 0.3% w/w.

In an embodiment, the diluent may be an organic diluent. In an embodiment, the diluent may be selected from paraffins (for example mineral oil fractions such as kerosene or diesel oil), coal tar oils and oils of vegetable or animal origin, aliphatic, cyclic and aromatic hydrocarbons, for example toluene, xylene, paraffin, tetrahydronaphthalene, alkylated naphthalenes or their derivatives, alcohols (for example methanol, butanol, pentanol, benzyl alcohol, cyclohexanol), ketones (for example cyclohexanone, gamma-butyrolactone), pyrrolidones (NMP, NEP, NOP), acetates (glycol diacetate), glycols, fatty acid dimethylamides, fatty acids and fatty acid esters, isophorone and dimethylsulfoxide. In an embodiment, the organic diluent may be present in an in an amount ranging from about 0% w/w to about 50% w/w of the total weight of the composition.

In an embodiment, the concentrated algal extract has a dry matter content of more than 5%, preferably about 5% to about 25%, about 6% to about 25%, about 8% to about 25%, about 10% to about 25%, about 12% to about 25%, about 14% to about 25%, about 16% to about 25%, about 18% to about 25%, more preferably about 20% to about 25%, wherein the dry matter is w/w of the total weight of the extract.

In an embodiment, the dry matter content of the concentrated algal extract in the composition is more than about 5%, preferably about 5% to about 25%, about 6% to about 25%, about 8% to about 25%, about 10% to about 25%, about 12% to about 25%, about 14% to about 25%, about 16% to about 25%, about 18% to about 25%, more preferably about 20% to about 25%, wherein the dry matter is w/w of the total weight of the composition.

The dry matter in the extract or the composition may be analysed by any conventional methods or any other methods known to a person skilled in the art. Such methods include analysis by moisture analyzers (thermobalance) such as, but not limited to, Sartorius MA35 Series Moisture Analyzer.

The term “dry matter” includes any one or both of inorganic components and organic components. The dry matter is usually measured using available technologies such as thermobalance or any other conventional methods known to a person skilled in the art.

In an embodiment, the concentrated algal extract has a total organic content of more than 5%. In an embodiment, the concentrated algal extract has a total organic content of about 5% to about 100%, about 5% to about 90%, about 5% to about 80%, about 5% to about 70%, about 5% to about 60%, about 5% to about 50%, about 5% to about 40%, about 5% to about 30% and more preferably about 5% to about 20%, wherein the total organic content is w/w content of the total weight of the extract. In another preferred embodiment, the concentrated algal extract has a total organic content of about 11% to about 18%.

The total organic content in the extract or the composition may be analysed by any conventional methods, or any other methods known to a person skilled in the art. Such methods include analysis by elemental analyzers such as, but not limited to, Thermoscientific, FlashSmart model. The total organic content is calculated as a measure of total carbon content. The formula used for calculation is as follows:

Total organic content=Total carbon content×2

In an embodiment, the composition of the present disclosure additionally comprises one or more agriculturally acceptable excipients. The agriculturally acceptable excipient is selected from any or a combination of wetting agent, dispersing agent, binding agent, diluent, solvent, surfactant/emulsifier, anti-freezing agent, mineral, filler, stabilizer, anti-foam and preservative. However, it should be appreciated that any other agriculturally acceptable excipients, as known to a person skilled in the art, may be used to serve its intended purpose without departing from the scope of the present disclosure.

In another embodiment, the compositions of the present disclosure can be presented in solid form, especially in the form of powders, dusts or granules, water dispersible granules, microcapsules or wettable powders or in liquid form, for example, emulsifiable concentrates, solutions, emulsions or suspensions, ZC formulations, oil dispersions.

An embodiment of the present disclosure provides a composition comprising a concentrated Ascophyllum nodosum extract, a preservative and optionally, a diluent; wherein the concentrated Ascophyllum nodosum extract is present in an amount ranging from about 50% w/w to about 99.75% w/w of the total weight of the composition, the concentration of the preservative is 0.15 w/w to 50% w/w of the total weight of the composition and the diluent is present in an in an amount ranging from about 0% w/w to about 50% w/w of the total weight of the composition.

An embodiment of the present disclosure provides a process of preparation of the compositions of the present disclosure. The steps to prepare compositions of the present disclosure comprise
i) adding a preservative in an amount of about 0.15% w/w to about 50% w/w to the concentrated algal extract which is present in an amount of about 50% w/w to about 99.75% w/w, and
ii) optionally adding a diluent to the composition obtained in step i) in an amount from about 0% w/w to about 50% w/w.

In an embodiment, the composition thus obtained is filtered and packed. In an embodiment, the composition thus obtained may be centrifuged prior to filtration and packing.

In an embodiment, the concentrated Ascophyllum nodosum extract is characterized by presence of any one or more of biomarkers selected from the group consisting of
a) Mannitol present in an amount ranging from about 1.5% w/w to about 7% w/w of the total weight of the extract;
b) Fucoidans present in an amount ranging from about 0.4% w/w to about 1.5% w/w of the total weight of the extract;
c) Polyphenols present in an amount ranging from about 0.2% w/w to about 5% w/w of the total weight of the extract;
d) Glycine betaine present in an amount ranging from about 0.0001% w/w to about 0.005% w/w of the total weight of the extract; and
e) Laminine present in an amount ranging from about 0.01% w/w to about 0.05% w/w of the total weight of the extract.

In an embodiment, the concentrated Ascophyllum nodosum extract is characterized by the presence of polyphenols in an amount ranging from about 0.2% w/w to about 5% w/w of the total weight of the extract.

In an embodiment, the concentrated Ascophyllum nodosum extract is characterized by the presence of mannitol in an amount ranging from about 1.5% w/w to about 7% w/w of the total weight of the extract. In an embodiment, the mannitol content in the extract is about 2% w/w to about 6% w/w of the total weight of the extract. In an embodiment, the mannitol content in the extract is in the range of about 3.5% w/w to about 4.5% w/w of the total weight of the extract.

In an embodiment, the concentrated Ascophyllum nodosum extract is characterized by the presence of fucoidans in an amount ranging from about 0.4% w/w to about 1.5% w/w of the total weight of the extract.

In an embodiment, the concentrated Ascophyllum nodosum extract is characterized by the presence of glycine betaine in an amount ranging from about 0.0001% w/w to about 0.005% w/w of the total weight of the extract.

In an embodiment, the concentrated Ascophyllum nodosum extract is characterized by the presence of laminine in an amount ranging from about 0.01% w/w to about 0.05% w/w of the total weight of the extract.

In an embodiment, the concentrated Ascophyllum nodosum extract is an acidic extract with a pH ranging between 2 and 4.

In an embodiment, the concentrated algal extract is prepared by a process comprising
i) crushing the algal cells to cause cell lysis;
ii) homogenizing the lysed algal cells in an aqueous solution;
iii) adding an acid to the homogenized lysed algal cells in an aqueous solution;
iv) removing alginate and cellulose by acidic flocculation and filtration; and
v) concentrating the filtrate to obtain the concentrated algal extract having a pH ranging between 2 and 4.

In an embodiment, the acid is selected from a weak acid, mild acid and strong acid; more preferably the acid is a weak acid or mild acid. In an embodiment, the weak or mild acid is selected from organic acids, oxalic acid, sulfurous acid, phosphoric acid, hydrogen sulfurous ion, nitrous acid, hydrofluoric acid, methanoic acid, benzoic acid, acetic acid, formic acid, citric acid, tartaric acid, ascorbic acid, lactic acid, uric acid, malic acid, succinic acid, propionic acid, acrylic acid, glyceric acid, carbonic acid, glycolic acid, glyoxylic acid, water, conjugates of weak bases, hydrocyanic and combinations thereof. In an embodiment, the organic acid is characterized by a carboxyl (—COOH) functional group, wherein the number of carbon atoms in the organic acid is C₁ to C₂₆.

In a preferred embodiment, the acid is a weak acid or a mild acid. Preferably, the acid is a weak acid or a mild acid such as citric acid, acetic acid, or combinations thereof.

In an embodiment, the acidic flocculation is carried presence of a strong acid selected from hydrochloric acid, sulphuric acid, perchloric acid and combinations thereof. In a preferred embodiment, the acidic flocculation is carried in presence of hydrochloric acid.

In an embodiment, the extract obtained has a dry matter content of more than about 5%, preferably about 5% to about 25%, about 6% to about 25%, about 8% to about 25%, about 10% to about 25%, about 11% to about 25%, about 12% to about 25%, about 14% to about 25%, about 16% to about 25%, about 18% to about 25%, more preferably about 20% to about 25%, wherein the dry matter is w/w content of the total weight of the extract.

In an embodiment, the extract obtained has a total organic content of about 5% to about 100%, about 5% to about 90%, about 5% to about 80%, about 5% to about 70%, about 5% to about 60%, about 5% to about 50%, about 5% to about 40%, about 5% to about 30% and more preferably about 5% to about 20%, even more preferably about 11% to about 18% wherein the total organic content is w/w content of the total weight of the extract.

The process of preparing a concentrated algal extract is also disclosed in the FR3025699. The present process differs from this prior art in step iii) wherein an acid, preferably a weak acid or a mild acid is added prior to acidic flocculation for adjusting pH and/or as a preservative, further concentrated to obtain the algal extract having a pH ranging between 2 and 4. In a preferred embodiment, said acid is citric acid or acetic acid or a combination thereof. In another preferred embodiment, the amount of citric acid or acetic acid or a combination thereof added is in the range of about 0.1% w/w to about 3% w/w.

In an embodiment, the extract has a pH in the range of 2 to 3, 3 to 4, or 2.1 to 3.2.

In an embodiment the present disclosure provides a concentrated algal extract obtained by a process comprising
i) crushing the algal cells to cause cell lysis;
ii) homogenizing the lysed algal cells in an aqueous solution;
iii) adding an acid to the homogenized lysed algal cells in an aqueous solution;
iv) removing alginate and cellulose by acidic flocculation and filtration; and
v) concentrating the filtrate to obtain the concentrated algal extract having a pH ranging between 2 and 4.

In an embodiment, the concentrated algal extract is an acidic extract with a pH ranging between 2 and 4, wherein the extract is acidified using an acid.

In an embodiment, the concentrated Ascophyllum nodosum extract is prepared by a process comprising
i) crushing the algal cells to cause cell lysis;
ii) homogenizing the lysed algal cells in an aqueous solution;
iii) adding an acid to the homogenized lysed algal cells in an aqueous solution;
iv) removing alginate and cellulose by acidic flocculation and filtration; and
v) concentrating the filtrate to obtain the concentrated algal extract having a pH ranging between 2 and 4.

In an embodiment the present disclosure provides a concentrated Ascophyllum nodosum extract obtained by a process comprising
i) crushing the algal cells to cause cell lysis;
ii) homogenizing the lysed algal cells in an aqueous solution;
iii) adding an acid to the homogenized lysed algal cells in an aqueous solution;
iv) removing alginate and cellulose by acidic flocculation and filtration; and
v) concentrating the filtrate to obtain the concentrated algal extract having a pH ranging between 2 and 4.

The composition comprising the concentrated algal extract according to the present disclosure is advantageously used in agriculture where it finds many applications depending on the type of plant on which it is applied and the period of the cycle of the plant where it is used.

The term “plant” or “target plant” as used herein refers to any vegetation to which the compositions of the present disclosure can be applied for any purposes or any treatment. The term covers a whole plant or a part thereof, such as stems, branches, bark, pistils, flowers, petals, sepals, roots, rhizomes, buds, bulbs, tubers, petioles, nodes, internodes, leaves, leaflets, meristems, root tips, shoot tips, fruits, reproductive organs such as anther, stamen, carpel, ovary, style, stigma, and so forth. The term also covers seeds, seedlings, or any plant propagation material. The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting. In an embodiment, the term includes agronomically useful plants, for example for example vegetable, fruit and cereal crops, and ornamental plants. The agronomically useful plants are angiosperms selected from the group consisting of Apiaceae, Asteraceae, Brassicaceae, Chenopodiaceae, Convolvulaceae, Cucurbitaceae, Fabaceae, Gramineae, Liliaceae, Polygonaceae, Rosaceae, Solanaceae, Poaceae, the Vitaceae. The term includes crops, cereals, fruits, vegetables, nuts, vines, nursery plants and flowers. Non-limiting examples of target plant include corn, cereals such as rice, wheat, barley, rye, oat, sorghum, millet, triticale, buckwheat, etc.; cotton, soybean, beet, row crops, legumes, grams, sugar cane, tobacco, etc.; oilseeds such as oilseed rape, peanut/groundnut, rapeseed, sunflower, etc.; vegetables: solanaceous vegetables such as eggplant, tomato, pimento, pepper, potato, etc., cucurbit vegetables such as cucumber, pumpkin, zucchini, water melon, melon, squash, gourds, muskmelon, etc., cruciferous vegetables such as radish, white turnip, horseradish, kohlrabi, cabbage, leaf mustard, broccoli, cauliflower, brussels sprouts, kale, etc., asteraceous vegetables such as burdock, crown daisy, artichoke, lettuce, etc, liliaceous vegetables such as green onion, onion, shallot, garlic, and asparagus, ammiaceous vegetables such as carrot, parsley, celery, parsnip, leek, etc., chenopodiaceous vegetables such as spinach, swiss chard, etc., lamiaceous vegetables such as Perilla frutescens, mint, basil, etc.; herbs and spices such as coriander, chamomile, Cassia, catnip, clove, cumin, curry, cilantro, cinnamon, cardamom, dill, anise, juniper, lavender, parsley, rosemary, marigold, mustard, nutmeg, fennel, poppy, thyme, vanilla, saffron, poppy, sage, wintergreen, etc.; flowers, foliage plants, turf grasses, fruits: pome fruits such apple, pear, quince, guava, etc, stone fleshy fruits such as peach, plum, nectarine, cherry, apricot, prune, etc., citrus fruits such as orange, lemon, lime, grapefruit, mandarin, malta, kumquat, pummelo, tangerine, tangor, uniq, etc., nuts such as chestnuts, walnuts, hazelnuts, almond, pistachio, cashew nuts, macadamia nuts, pecan nut, cashew nut, hazel nut, pine nut, etc. berries such as caneberry, strawberry, blueberry, cranberry, blackberry, raspberry, coryberry, darrowberry, dewberry, thornless berry, evergreen blackberry, himalayaberry, hullberry, lavacaberry, loganberry, lowberry, lucretiaberry, mammoth blackberry, marionberry, mora, mures deronce, nectarberry, olallieberry, evergreen berry, phenomenalberry, rangeberry, ravenberry, rossberry, dewberry, tayberry, youngberry, zarzamora, Aronia berry, currant, elderberry, barberry, gooseberry, honeysuckle, huckleberry, jostaberry, juneberry, lingonberry, salal, seabuckthorn, bayberry, buffaloberry, chokecherry, maypop, mulberry, bearberry, bilberry, cloudberry, muntries, partridgeberry, etc., grape, kaki fruit, kiwi fruit, olive, plum, banana, coffee, date palm, coconuts, papaya, persimmon, avocado, dragon fruit, pomegranate, lychee, jackfruit, pineapple, passionfruit, sapota, etc., trees other than fruit trees; tea, mulberry, flowering plant, trees such as ash, birch, dogwood, Eucalyptus, Ginkgo biloba, lilac, maple, Quercus, poplar, Judas tree, Liquidambar formosana, plane tree, Zelkova, japanese arborvitae, fir wood, hemlock, juniper, Pinus, Picea, and Taxus cuspidate, etc., other crops such as chive, day lily, Elegans hosta, Fritillaria, gojiberry, okra, pea, hops, beans, guar, radish, amaranth, jute, fenugreek, lentils, chickpea, artichoke, rhubarb, licorice, sweet potato, Dioscorea japonica, colocasia, ornamental grasses (lawn turf, sod, etc.), varieties and cultivars thereof.

In an embodiment, the present disclosure provides a method of growth stimulation in a target plant, wherein the method comprises applying to the target plant or a locus a composition comprising a concentrated algal extract, a preservative and optionally, a diluent; wherein the concentrated algal extract is present in an amount ranging from about 50% w/w to about 99.75% w/w of the total weight of the composition, the concentration of the preservative is 0.15 w/w to 50% w/w of the total weight of the composition and the diluent is present in an in an amount ranging from about 0% w/w to about 50% w/w of the total weight of the composition.

The term “locus” as used herein shall denote the vicinity of the target plant in which growth stimulation is desired. The locus includes the vicinity of the target plant which has either emerged or is yet to emerge. The term “target plant” shall include a multitude of desired crop plants or an individual crop plant growing at a locus. The said locus includes the area, medium and soil where the target plant grows.

The term “growth stimulation” as used herein refers to any improvement in plant health, phenotypic or genotypic characteristics of a plant; visible beneficial changes in a plant; improvement in plant stress and disease tolerance and resistance and improvement in yield and quality of produce. Non-limiting examples of growth stimulation include:
Improved plant nutrient use efficiency;
Enhanced root and shoot growth;
Improved reproductive heat stress tolerance;
Improved drought tolerance;
Improved pollen tube growth;
Enhanced pollen viability;
Increased fertilization and fruit set;
Increased floral inflorescence primordia;
Increased number of buds, pods and yield;
Improved and strong root mass and architecture;
Improved/increased bud development;
Accelerated shoot or bud emergence;
Enhanced vigor/uniformity of emergence;
Improved and increased branching;
Improved/increased diameter and strength;
Improved/increased inter-node length;
Increase in number of yield structures (ears, fruits etc);
Increase in leaf area;
Increased amount of chlorophyll, greening;
Increased photosynthesis activity;
Increase in CO₂ fixation;
Accelerated and improved flowering;
Improvement in pollination;
Enhanced fruit set & retention;
Improvement in cell division for size and quality potential;
Improvement in fruit finish;
Freedom from pest such as fungal pests, viruses, bacteria, weeds, insects, nematodes;
Enhanced resistance against pest such as fungal pests, viruses, bacteria, weeds, insects, nematodes;
Increased plant weight;
Increased plant height;
Increased biomass such as higher overall fresh weight;
Higher grain yield;
More tillers;
Larger leaves;
Increased shoot growth;
Increased protein content;
Increased oil content;
Increased starch content;
Increased pigment content;
Increased plant vigor;
Improved vitality of the plant;
Improved plant growth;
Improved plant development;
Improved visual appearance of the plant or a part thereof;
Improved plant stand (less plant verse/lodging);
Improved emergence;
Enhanced nodulation, in particular rhizobial nodulation;
Bigger leaf blade;
Increased yield when grown on poor soils or unfavorable climate;
Enhanced pigment content (e.g. Chlorophyll content);
Earlier flowering;
Earlier fruiting;
Earlier and improved germination;
Earlier grain maturity;
Improved self-defence mechanisms;
Improved stress tolerance and resistance of the plants against biotic and abiotic stress factors such as fungi, bacteria, viruses, insects, heat stress, cold stress, drought stress, UV stress and/or salt stress;
Less non-productive tillers;
Less dead basal leaves;
Less input needed (such as fertilizers or water);
Greener leaves;
Complete maturation under shortened vegetation periods;
Less fertilizers needed;
Less seeds needed;
Easier harvesting;
Faster and more uniform ripening;
Longer shelf-life;
Longer panicles;
Delay of senescence;
Stronger and/or more productive tillers;
Better extractability of ingredients;
Improved quality of seeds (for being seeded in the following seasons for seed production);
Reduced production of ethylene and/or the inhibition of its reception by the plant;
Increased nutrient content;
Increased content of fatty acids;
Increased metabolite content;
Increased carotenoid content;
Increased sugar content;
Increased amount of essential amino acids·improved nutrient composition;
Improved protein composition;
Improved composition of fatty acids;
Improved metabolite composition;
Improved carotenoid composition;
Improved sugar composition;
Improved amino acids composition;
Improved or optimal fruit color;
Improved leaf color;
Higher storage capacity; and/or
Higher processability of the harvested products.

In an embodiment, the present disclosure provides a method of increasing growth in a target plant, wherein the method comprises applying to the target plant or a locus a composition comprising a concentrated algal extract, a preservative and optionally, a diluent; wherein the concentrated algal extract is present in an amount ranging from about 50% w/w to about 99.75% w/w of the total weight of the composition, the concentration of the preservative is 0.15 w/w to 50% w/w of the total weight of the composition and the diluent is present in an in an amount ranging from about 0% w/w to about 50% w/w of the total weight of the composition.

In an embodiment, the present disclosure provides a method of increasing yield of a target plant, wherein the method comprises applying to the target plant or a locus a composition comprising a concentrated algal extract, a preservative and optionally, a diluent; wherein the concentrated algal extract is present in an amount ranging from about 50% w/w to about 99.75% w/w of the total weight of the composition, the concentration of the preservative is 0.15 w/w to 50% w/w of the total weight of the composition and the diluent is present in an in an amount ranging from about 0% w/w to about 50% w/w of the total weight of the composition.

It was also observed that compositions of the present disclosure can promote growth of plant and increase in yield of plant in reduced nutrient or nutrient deficient conditions. Thus, the compositions of the present disclosure help in reducing dependency on chemical fertilizers, thus promoting a sustainable and environmentally friendly way of growing plants, especially agricultural plants and crops and increasing their yield.

Thus, in an embodiment, the present disclosure provides a method of promoting growth and increasing yield of a target plant in nutrient deficient conditions, wherein the method comprises applying to the target plant or a locus a composition comprising a concentrated algal extract, a preservative and optionally, a diluent; wherein the concentrated algal extract is present in an amount ranging from about 50% w/w to about 99.75% w/w of the total weight of the composition, the concentration of the preservative is 0.15 w/w to 50% w/w of the total weight of the composition and the diluent is present in an in an amount ranging from about 0% w/w to about 50% w/w of the total weight of the composition.

An embodiment of the present disclosure also provides a method of determining the quality of the composition of the present disclosure, said method comprising
i) obtaining a sample of said composition; and
ii) measuring the pH and the concentration of any one or more of biomarkers selected from mannitol, glycine betaine, fucoidans, polyphenols and laminine in the sample of said composition;
wherein said composition is considered stable and good provided the pH of the sample of said composition is detected to be in a range of 2 to 4 and wherein the concentration of any one or more of biomarkers selected from mannitol, glycine betaine, fucoidans, polyphenols and laminine are detected to be in a range as provided in Table 2.

The plants on which the compositions comprising the concentrated algal extract of the present disclosure can be used are agronomically useful plants, for example for example vegetable, fruit and cereal crops, and ornamental plants. The agronomically useful plants are angiosperms selected from the group consisting of Apiaceae, Asteraceae, Brassicaceae, Chenopodiaceae, Convolvulaceae, Cucurbitaceae, Fabaceae, Gramineae, Liliaceae, Polygonaceae, Rosaceae, Solanaceae, Poaceae, the Vitaceae. Thus, in an embodiment, the target plant is selected from crops, cereals, fruits, vegetables, nuts, vines, nursery plants and flowers. In an embodiment, non-limiting examples of target plant include corn, cereals such as rice, wheat, barley, rye, oat, sorghum, millet, triticale, buckwheat, etc.; cotton, soybean, beet, row crops, legumes, grams, sugar cane, tobacco, etc.; oilseeds such as oilseed rape, peanut/groundnut, rapeseed, sunflower, etc.; vegetables: solanaceous vegetables such as eggplant, tomato, pimento, pepper, potato, etc., cucurbit vegetables such as cucumber, pumpkin, zucchini, water melon, melon, squash, gourds, muskmelon, etc., cruciferous vegetables such as radish, white turnip, horseradish, kohlrabi, cabbage, leaf mustard, broccoli, cauliflower, brussels sprouts, kale, etc., asteraceous vegetables such as burdock, crown daisy, artichoke, lettuce, etc, liliaceous vegetables such as green onion, onion, shallot, garlic, and asparagus, ammiaceous vegetables such as carrot, parsley, celery, parsnip, leek, etc., chenopodiaceous vegetables such as spinach, swiss chard, etc., lamiaceous vegetables such as Perilla frutescens, mint, basil, etc.; herbs and spices such as coriander, chamomile, Cassia, catnip, clove, cumin, curry, cilantro, cinnamon, cardamom, dill, anise, juniper, lavender, parsley, rosemary, marigold, mustard, nutmeg, fennel, poppy, thyme, vanilla, saffron, poppy, sage, wintergreen, etc.; flowers, foliage plants, turf grasses, fruits: pome fruits such apple, pear, quince, guava, etc, stone fleshy fruits such as peach, plum, nectarine, cherry, apricot, prune, etc., citrus fruits such as orange, lemon, lime, grapefruit, mandarin, malta, kumquat, pummelo, tangerine, tangor, uniq, etc., nuts such as chestnuts, walnuts, hazelnuts, almond, pistachio, cashew nuts, macadamia nuts, pecan nut, cashew nut, hazel nut, pine nut, etc. berries such as caneberry, strawberry, blueberry, cranberry, blackberry, raspberry, coryberry, darrowberry, dewberry, thornless berry, evergreen blackberry, himalayaberry, hullberry, lavacaberry, loganberry, lowberry, lucretiaberry, mammoth blackberry, marionberry, mora, mures deronce, nectarberry, olallieberry, evergreen berry, phenomenalberry, rangeberry, ravenberry, rossberry, dewberry, tayberry, youngberry, zarzamora, Aronia berry, currant, elderberry, barberry, gooseberry, honeysuckle, huckleberry, jostaberry, juneberry, lingonberry, salal, seabuckthorn, bayberry, buffaloberry, chokecherry, maypop, mulberry, bearberry, bilberry, cloudberry, muntries, partridgeberry, etc., grape, kaki fruit, kiwi fruit, olive, plum, banana, coffee, date palm, coconuts, papaya, persimmon, avocado, dragon fruit, pomegranate, lychee, jackfruit, pineapple, passionfruit, sapota, etc., trees other than fruit trees; tea, mulberry, flowering plant, trees such as ash, birch, dogwood, Eucalyptus, Ginkgo biloba, lilac, maple, Quercus, poplar, Judas tree, Liquidambar formosana, plane tree, Zelkova, japanese arborvitae, fir wood, hemlock, juniper, Pinus, Picea, and Taxus cuspidate, etc., other crops such as chive, day lily, Elegans hosta, Fritillaria, gojiberry, okra, pea, hops, beans, guar, radish, amaranth, jute, fenugreek, lentils, chickpea, artichoke, rhubarb, licorice, sweet potato, Dioscorea japonica, colocasia, ornamental grasses (lawn turf, sod, etc.), varieties and cultivars thereof.

In a preferred embodiment, the target plant is corn, row crops, legumes, cereal, rice, oilseed rape, oilseeds, soybean, tomato, potato, wheat, cotton, cherry, apple, grape, wine grape, citrus, pome fruit, fruiting vegetables, almond, pecan nut, walnut, pistachio and peanut/groundnut. An embodiment also provides a seed coated with the compositions of the present disclosure.

The compositions of the present disclosure as per an embodiment can be applied
Pre-emergence.
Post-emergence.
Before sowing
Pre-harvest
Post-harvest

In an embodiment, the compositions of the present disclosure is applied before planting/sowing; at the time of sowing, germination, early leaf stage, early bloom stage, pre-bloom stage, full bloom stage, petal fall stage, swollen bud stage, bud break stage, post petal fall stage, post bud break stage, pre-bud break stage, full pistillate stage, post pollination stage, square formation stage, pegging stage, post-pegging stage, fruit set stage, early mid-bloom, greenup (post-dormancy) stage, bud set stage, etc; after flowering stage; harvest stage; and/or post-harvest. In an embodiment, the compositions of the present disclosure may be applied anytime throughout the growth stage as conventionally known to a person skilled in the art. The time of application depends on the target plant; environmental conditions such as nutrient deficiency, biotic and abiotic stresses; type of application and the expected outcome or any other parameter known to a person skilled in the art. Applications can be made throughout the growth of the plant, one or more times a week.

In an embodiment, the composition of the present invention is applied directly and/or indirectly to the plant and/or to plant propagation material by drenching the soil, by drip application onto the soil, by soil injection, by dipping or by treatment of seeds.

The compositions of the present disclosure may be applied by dusting, spraying, granular application, seed pelleting/seed dressing, broadcasting, in furrow application, side dressing, spot application, ring application, root zone application, pralinage, seedling root dip, sett treatment, trunk/stem injection, padding, swabbing, root feeding, soil drenching, capsular placement, baiting, fumigation, banding, foliar application, basal application, space treatment, enclosed space fumigation and such other methods which may help prevent or control or eradicate the disease.

In a preferred embodiment, the compositions of the present disclosure are applied as a drench application, in-furrow application, soil, drip irrigation, soil injection, hydroponic application, capillary action application, root infiltration, or a foliar application. In an embodiment, the compositions of the present disclosure is applied as a seed treatment such as seed soak application, seed coating application, germination treatment; or a rooting/shooting dip treatment. The type of application is decided as per the target plant, the outcome expected, and the time of application and any other conventional parameters as known to a person skilled in the art.

The present compositions can be applied by the use of conventional ground sprayers, granule applicators, watering (drenching), drip irrigation, spraying, atomizing, broadcasting, dusting, foaming, spreading-on, aerial methods of spraying, aerial methods of application, methods utilizing application using modern technologies such as, but not limited to, drones, robots, predosage device, a knapsack sprayer, a spray tank, a spray plane, or an irrigation system and by other conventional means known to those skilled in the art.

In an embodiment, the compositions of the present disclosure is applied at a rate of 10 ml/ha to 1000 ml/ha. In an embodiment, the compositions of the present disclosure is applied at a rate of 50 ml/ha to 500 ml/ha. In an embodiment, the compositions of the present disclosure is applied at a rate of 70 ml/ha to 300 ml/ha. In an embodiment, the compositions of the present disclosure is applied at a rate of 30 ml/ha to 600 ml/ha.

In an embodiment, the compositions of the present disclosure is applied at a rate of 10 ml/1 kg seed to about 700 ml/1 kg seed.

In an embodiment, the compositions of the present disclosure is applied at a rate of 10 ml/100 kg seed to about 700 ml/100 kg seed.

In an embodiment, the dose rate of the compositions of the present disclosure ranges from about 0.01 ml/L to about 100 ml/L. In a preferred embodiment, the dose rate of the compositions of the present disclosure ranges from about 0.1 ml/L to about 50 ml/L. In another preferred embodiment, the dose rate of the compositions of the present disclosure ranges from about 0.2 ml/L to about 10 ml/L. In another preferred embodiment, the dose rate of the compositions of the present disclosure ranges from about 0.25 ml/L to about 1 ml/L.

The compositions comprising concentrated algal extract of the present disclosure can in particular be used alone or in combination with a nutritional, biostimulant or phytosanitary composition.

In a further embodiment, the compositions of the present disclosure are combined with, formulated with and/or applied with one or more additional agrochemically active substances. According to an embodiment, the said agrochemically active substance is selected from fertilizers, mycorrhiza, micronutrients, acaricides, algicides, antifeedants, avicides, bactericides, bird repellents, chemosterilants, fungicides, herbicide safeners, herbicides, insect attractants, insect repellents, insecticides, mammal repellents, mating disruptors, molluscicides, nematicides, plant activators, plant-growth regulators, rodenticides, synergists, virucides, derivatives thereof, biological control agents, superabsorbent polymers and mixtures thereof.

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### EXAMPLES

### Example 1: Composition Comprising Ascophyllum nodosum Extract, Preservative and Diluent

TABLE 1
Composition comprising Ascophyllum nodosum
extract, preservative and diluent

| | Content in |
|---|---|
| Ingredient | % by weight |
| Ascophyllum nodosum | 99.52 |
| Concentrated Extract | |
| Potassium Sorbate | 0.23 |
| Water | 0.25 |
| Total | 100 |

Process of Preparation of Concentrated Algal Extract of Ascophyllum nodosum:

Fresh brown seaweed Ascophyllum nodosum was harvested from ocean waters. The seaweed was subjected to mechanical form of crushing followed by micro-crushing to lyse the algal cells. 0.5% w/w citric acid (99% purity) was added to the crushed and homogenized seaweed (algal cells). The resulting solution was further subjected to flocculation in acidic conditions to remove alginates and cellulose. The flocculated solution was subjected to filtration. The filtrate was concentrated through vacuum evaporation to obtain the concentrated algal extract of Ascophyllum nodosum. pH of the concentrated extract was measured using a pH meter (Mettler Toledo, SevenCompact). The pH was found to be in range of 2 to 4.

The above concentrated algal extract is characterized by following biomarkers. The biomarkers were analyzed by LC/MS/MS (liquid chromatography coupled with mass spectrometry detection) using standard protocol.

TABLE 2
Characterization of the concentrated algal extract
of Ascophyllum nodosum by biomarkers.

| | Content in |
|---|---|
| Biomarker | % by weight |
| Mannitol | 1.5 to 7 |
| Fucoidans | 0.4 to 1.5 |
| Polyphenols | 0.2 to 5 |
| Glycine betaine | 0.0001 to 0.005 |
| Laminine | 0.01 to 0.05 |

Process of Preparation of Composition of Concentrated Algal Extract of Ascophyllum nodosum:

0.23% w/w of potassium sorbate was added to 99.52% w/w of the concentrated algal extract of Ascophyllum nodosum which was further mixed with diluent to obtain the final composition as provided in Table 1.

The final composition as provided in Table 1 was again subjected pH and biomarker analysis conducted as provided herein. The pH of the final composition was found to be in a range of 2 to 4 and the biomarker concentrations were detected to be in range as provided in Table 2.

### Example 2: Dry Matter Content and Total Organic Content/Total Carbon Content of Ascophyllum nodosum Concentrated Extract and Composition of Example 1 (Table 1)

Dry matter content of Ascophyllum nodosum Concentrated Extract and composition of Example 1 (Table 1) was determined using thermobalance (Sartorius MA35 Series Moisture Analyzer).

Dry matter content of Ascophyllum nodosum Concentrated Extract and composition of Example 1 (Table 1) was found to be in the range of 20% to 25%.

An elemental analyser Thermoscientific-FlashSmart model was used to measure carbon content of the Ascophyllum nodosum Concentrated Extract and composition of Example 1 (Table 1).

Organic matter content was calculated by following formula

$$Organic matter content = Carbon content ( Ascophyllum Nodosum Concentrated Extract ) \times 2$$

The organic matter content of the Ascophyllum nodosum Concentrated Extract and composition of Example 1 (Table 1) was found to be in the range of 11% to 18%.

### Example 3: Stability Evaluation for Composition of Example 1

The composition of Example 1 was subjected to stability evaluation for a period of 36 months. Parameters selected for evaluation of the stability of the composition of Example 1 were pH, mannitol content and foaming. The composition of Example 1 was stored for 2 weeks (54° C.), 2 months (−10° C., 40° C.), 6 months (20° C.), 12 months (20° C.), 24 months (20° C.) and 36 months (20° C.). A zero-day reading of evaluation parameters at room temperature was used for comparison.

TABLE 3
Storage stability of composition of Example 1 for
a period of 36 months at varying temperatures

| | | | | | Mannitol | |
|---|---|---|---|---|---|---|
| | | | | | content | |
| Storage | | Storage | | pH | (3.5% w/w to | Persistent |
| time | | temperature | | (2 to 4) | 4.5% w/w) | foam |
| Zero-day | | Room | | Found to | Found to | None |
| (initial) | | temperature | | be in the | be in the | |
| | | | | given range | given range | |
| 2 | weeks | 54° | C. | Found to | Found to | None |
| | | | | be in the | be in the | |
| | | | | given range | given range | |
| 2 | months | −10° | C. | Found to | Found to | None |
| | | | | be in the | be in the | |
| | | | | given range | given range | |
| 2 | months | 40° | C. | Found to | Found to | None |
| | | | | be in the | be in the | |
| | | | | given range | given range | |
| 6 | months | 20° | C. | Found to | Found to | None |
| | | | | be in the | be in the | |
| | | | | given range | given range | |
| 12 | months | 20° | C. | Found to | Found to | None |
| | | | | be in the | be in the | |
| | | | | given range | given range | |
| 24 | months | 20° | C. | Found to | Found to | None |
| | | | | be in the | be in the | |
| | | | | given range | given range | |
| 36 | months | 20° | C. | Found to | Found to | None |
| | | | | be in the | be in the | |
| | | | | given range | given range | |

It can be observed that the composition of Example 1 remained stable throughout its storage period at different temperatures. The samples without preservative were found to be discolored and degraded in the same span of time.

### Example 4: Composition Comprising Ascophyllum nodosum Extract and Preservatives

TABLE 4
Composition comprising Ascophyllum nodosum
extract and two preservatives

| | Content in |
|---|---|
| Ingredient | % by weight |
| Ascophyllum Nodosum | 99.52 |
| Concentrated Extract | |
| Potassium Sorbate | 0.23 |
| Sodium metabisulphite | 0.25 |
| Total | 100 |

Process of Preparation of Concentrated Algal Extract of Ascophyllum nodosum:

Fresh brown seaweed Ascophyllum nodosum was harvested from ocean waters. The seaweed was subjected to mechanical form of crushing followed by micro-crushing to lyse the algal cells. 0.5% w/w citric acid (99% purity) was added to the crushed and homogenized seaweed and subjected to flocculation in acidic conditions. The flocculated solution was subjected to filtration. The filtrate was concentrated through vacuum evaporation to obtain the concentrated algal extract of Ascophyllum nodosum.

Process of Preparation of Composition of Concentrated Algal Extract of Ascophyllum nodosum:

0.23% w/w of potassium sorbate and 0.25% w/w of sodium metabisulphite was added to 99.52% w/w of the concentrated algal extract of Ascophyllum nodosum to obtain the final composition as provided in Table 4. The composition of Table 4 is also observed to be stable and efficacious.

The composition of Table 4 was analysed for its pH using a pH meter (Mettler Toledo, SevenCompact), dry matter using a thermobalance (Sartorius MA35), and organic matter content was obtained by measuring % carbon (C) content using elemental analyzer (ThermoScientific, FlashSmart) and then multiplied by a factor of 2 to get % organic content. The analysis was conducted in triplicate. The observations are recorded in Table 5 below:

TABLE 5
pH, dry matter content and organic
matter content of composition of Table 4
Composition of Table 4

pH: in range of 2 to 4
Dry matter: in range of 20% w/w to 25% w/w
Organic matter: in range of 11% w/w to 18% w/w

Biomarker analysis of the composition of Table 4 was carried out for a period of 12 months by LC/MS/MS (liquid chromatography coupled with mass spectrometry detection) using standard protocol. The analysis was conducted in two sets. The observations are recorded below in Table 6.

TABLE 6
Biomarker analysis of composition of
Table 4 for a period of 12 months.

| | | | | Betaine | Laminine | Poly- |
|---|---|---|---|---|---|---|
| Duration of | | Mannitol | Fucoidan | 0.0001 | 0.01 | phenols |
| storage of | | 1.5 to 7 | 0.4 to 1.5 | to 0.005 | to 0.05 | 0.2 to 5 |
| sample | | (% w/w) | (% w/w) | (% w/w) | (% w/w) | (% w/w) |
| Time 0 | | Found | Found | Found | Found | Found |
| | | to be | to be | to be | to be | to be |
| | | in range | in range | in range | in range | in range |
| 3 | Months | Found | Found | Found | Found | Found |
| | | to be | to be | to be | to be | to be |
| | | in range | in range | in range | in range | in range |
| 6 | Months | Found | Found | Found | Found | Found |
| | | to be | to be | to be | to be | to be |
| | | in range | in range | in range | in range | in range |
| 9 | Months | Found | Found | Found | Found | Found |
| | | to be | to be | to be | to be | to be |
| | | in range | in range | in range | in range | in range |
| 12 | Months | Found | Found | Found | Found | Found |
| | | to be | to be | to be | to be | to be |
| | | in range | in range | in range | in range | in range |

The samples without preservative were found to be discolored and degraded in the same span of time.

### Example 5: Use of the Composition of Example 1 in Corn for Increase in Yield

The composition of Example 1 was applied at rates of 73 ml/ha at V4 (fourth leaf collar visible) stage in corn. The trial was conducted in 6 sets. The trial was conducted in the year 2020 in USA.

The yield of corn obtained was measured in kg/ha (average of yield from 6 sets of trials).

TABLE 7
Corn yield due to application of composition of Example 1

| | Application | Yield |
|---|---|---|
| Treatment | rate (ml/ha) | (kg/hectare) |
| Untreated | — | 14073.63 |
| Example 1 | 73 | 14497.98 |

In the above trial, it was observed that there was 3% rise in the yield of corn with application of the composition of Example 1.

### Example 6: Use of Composition of Example 4 in Corn-Delayed Senescence and Yield Increase in Standard N Conditions and Reduced N Conditions

An in-furrow application of the composition of Example 4 in corn at rates 36.5 ml/kg of seed and 365 ml/kg of seed was carried out at the time of sowing in presence of standard N (nitrogen) conditions—Trial A, and reduced N (nitrogen) conditions—Trial B. The standard N conditions involved application of an NPK fertilizer in a standard dose of 120:60:40 (N:P:K kg/ha). The reduced N conditions involved application of an NPK fertilizer in a dose of 00:60:40 (N:P:K kg/ha). The parameters evaluated were number of days the onset of senescence began after sowing and cob weight (weight of 10 cobs in kg).
Trial A: standard N conditions—120:60:40 (N:P:K kg/ha)
Trial B: reduced N conditions—00:60:40 (N:P:K kg/ha)

### Observations:

### Trial A

TABLE 8
Effect of composition of Example 1 on senescence onset,
cob weight and kernels in corn in standard N conditions.

| | | Onset of | | |
|---|---|---|---|---|
| | Application | senescence | | Weight |
| | rate | (days after | Number of | of 10 |
| Treatment | (ml/kg seed) | sowing) | Kernels/Cob | Cob (Kg) |
| Untreated | — | 132.70 | 406.40 | 2.03 |
| composition | 36.5 | 144.20 | 463.13 | 2.31 |
| of Example 4 | | | | |
| composition | 365 | 148.00 | 481.88 | 2.41 |
| of Example 4 | | | | |

### Trial B

TABLE 9
Effect of composition of Example 4 on senescence onset,
cob weight and kernels in corn in reduced N conditions

| | | Onset of | | |
|---|---|---|---|---|
| | Application | senescence | | Weight |
| | rate | (days after | Number of | of 10 |
| Treatment | (ml/kg seed) | sowing) | Kernels/Cob | Cob (Kg) |
| Untreated | — | 124.20 | 380.32 | 1.83 |
| composition | 36.5 | 134.70 | 416.95 | 2.14 |
| of Example 4 | | | | |
| composition | 365 | 140.00 | 421.87 | 2.22 |
| of Example 4 | | | | |

It is thus observed that the composition of Example 4 delayed the onset of senescence in corn and produced cobs with more weight and kernels in comparison with untreated samples, in both standard as well as reduced nitrogen conditions.

### Example 7: Use of Composition of Example 4 in Corn for Increase in Shoot Weight, Root Weight and Vigor

An in-furrow application of the composition of Example 4 in corn at rates 36.5 ml/kg of seed, 365 ml/kg of seed were carried out at the time of sowing in presence of standard N (nitrogen) conditions—Trial A, and reduced N (nitrogen) conditions—Trial B. The standard N conditions involved application of an NPK fertilizer in a standard dose of 120:60:40 (N:P:K kg/ha). The reduced N conditions involved application of an NPK fertilizer in a dose of 00:60:40 (N:P:K kg/ha). The parameters evaluated were shoot fresh weight BBCH 12 stage, BBCH 14 stage and BBCH 51 stage; root fresh weight at BBCH 51 stage; vigor at BBCH 30 and BBCH 51 stage.
Trial A: standard N conditions—120:60:40 (N:P:K kg/ha)
Trial B: reduced N conditions—00:60:40 (N:P:K kg/ha)

BBCH (Biologische Bundesanstalt, Bundessortenamt und Chemische Industrie) scale is a system for uniform coding of growth stages which has been widely used for describing phenological stages of plants (Meier, U. (2001) Growth Stages of Mono and Dicotyledonous Plants. BBCH Monograph, Federal Biological Research Centre for Agriculture and Forestry, Bonn.)

### Observations

### Trial A

TABLE 10
Effect of composition of Example 1 on shoot fresh weight, root
fresh weight and vigor of corn in standard N conditions.

| | | | Corn treated with composition | |
|---|---|---|---|---|
| | | | of Example 4 at rates | |
| Observation | Evaluation | Untreated | 36.5 ml/kg | 365 ml/kg |
| Parameters | stage | Corn | of seed | of seed |
| Shoot fresh | BBCH 12 | 17.08 | 18.16 | 19.37 |
| weight (gm) | BBCH 14 | 56.76 | 67.85 | 72.85 |
| | BBCH 51 | 1204.86 | 1464.94 | 1592.21 |
| Root fresh | BBCH 51 | 493.47 | 589.90 | 633.42 |
| weight (gm) | | | | |
| Vigor | BBCH 30 | 5.00 | 6.00 | 7.00 |
| | BBCH 51 | 5.00 | 6.00 | 7.00 |

### Trial B

TABLE 11
Effect of composition of Example 4 on shoot fresh weight, root
fresh weight and vigor of corn in reduced N conditions.

| | | | Corn treated with composition | |
|---|---|---|---|---|
| | | | of Example 4 at rates | |
| Observation | Evaluation | Untreated | 36.5 ml/kg | 365 ml/kg |
| Parameters | stage | Corn | of seed | of seed |
| Shoot fresh | BBCH 12 | 13.40 | 14.89 | 15.08 |
| weight (gm) | BBCH 14 | 47.23 | 54.10 | 58.57 |
| | BBCH 51 | 966.37 | 1178.96 | 1237.16 |
| Root fresh | BBCH 51 | 412.36 | 539.22 | 583.25 |
| weight (gm) | | | | |
| Vigor | BBCH 30 | 5.00 | 5.50 | 6.00 |
| | BBCH 51 | 5.00 | 6.50 | 6.70 |

It is thus observed that the composition of Example 4 improved the shoot fresh weight, root fresh weight and vigor of plant at different stages of growth in comparison with untreated samples, in both standard as well as reduced nitrogen conditions.

### Example 8

An in-furrow application of the composition of Example 4 in corn at rates 36.5 ml/kg of seed, 365 ml/kg of seed and 600 ml/kg of seed were carried out at the time of sowing in presence of standard N (nitrogen) conditions—Trial A, and reduced N (nitrogen) conditions—Trial B. The standard N conditions involved application of an NPK fertilizer in a standard dose of 120:60:40 (N:P:K kg/ha). The reduced N conditions involved application of an NPK fertilizer in a dose of 00:60:40 (N:P:K kg/ha). The parameters evaluated were stem diameter at BBCH 51 stage, green index (%) at BBCH 30 and BBCH 51 stage; and cob yield per plot (kg).
Trial A: standard N conditions—120:60:40 (N:P:K kg/ha)
Trial B: reduced N conditions—00:60:40 (N:P:K kg/ha)

BBCH (Biologische Bundesanstalt, Bundessortenamt und Chemische Industrie) scale is a system for uniform coding of growth stages which has been widely used for describing phenological stages of plants (Meier, U. (2001) Growth Stages of Mono and Dicotyledonous Plants. BBCH Monograph, Federal Biological Research Centre for Agriculture and Forestry, Bonn.)

### Trial A

TABLE 12
Effect of composition of Example 1 on stem diameter, green
index and yield of cob per plot in standard N conditions

| | | | Corn treated with composition | | |
|---|---|---|---|---|---|
| | | | of Example 4 at rates | | |
| Observation | Evaluation | Untreated | 36.5 ml/kg | 365 ml/kg | 600 ml/kg |
| Parameters | stage | Corn | of seed | of seed | of seed |
| Stem | BBCH 51 | 22.05 | 24.32 | 25.43 | 25.78 |
| Diameter | | | | | |
| (mm) | | | | | |
| Green index | BBCH 30 | 72.50 | 79.20 | 81.70 | 83.30 |
| (%) | BBCH 51 | 82.50 | 90.00 | 93.30 | 94.20 |
| Yield of | | 15.98 | 17.85 | 18.12 | 18.72 |
| cobs/plot | | | | | |
| (kg) | | | | | |

### Trial B

TABLE 13
Effect of composition of Example 4 on stem diameter, green
index and yield of cob per plot in reduced N conditions.

| | | | Corn treated with composition | | |
|---|---|---|---|---|---|
| | | | of Example 4 at rates | | |
| Observation | Evaluation | Untreated | 36.5 ml/kg | 365 ml/kg | 600 ml/kg |
| Parameters | stage | Corn | of seed | of seed | of seed |
| Stem | BBCH 51 | 20.63 | 22.28 | 22.42 | 22.82 |
| Diameter | | | | | |
| (mm) | | | | | |
| Green index | BBCH 30 | 70.80 | 77.50 | 80.80 | 84.20 |
| (%) | BBCH 51 | 80.00 | 87.50 | 90.00 | 91.70 |
| Yield of | | 13.8 | 15.27 | 15.82 | 15.84 |
| cobs/plot | | | | | |
| (kg) | | | | | |

### Example 9: Effect of Application of Composition of Example 1 in Wheat

Wheat seeds were sown on 9 cm square pot containing vermiculite super fine. The pots were placed on a rotating tray in a growth chamber set on 20° C./18° C. (day/night; 16/8 h) and 70% relative humidity under a light intensity of 252±20 μmol·m⁻²·s⁻¹. Plants were watered with tap water until 8 days after sowing and then with a nutrient solution N/P/K 20:20:20 at 1 g/L every 2 days.

1 mL of composition of Example 1 was applied per pot. Five applications of products were carried out via root application. The application was carried out 15 days after sowing (DAS) and this every two days until the fifth soil application. The products were applied through root after having diluted the corresponding volume of product in a Plant Prod N/P/K 20:20:20 nutrient solution at 1 g/L, the volume being adapted following the plant's need.

An untreated control was set up wherein the composition of Example 1 was not applied.

Wheat plants were harvested at 28 days after sowing. The aerial parts were cut, and their fresh weight (FW) was obtained by weighing this samples on a 0.01 g precision weighing scale (Provider: Radwag PS 6000/X). Plant aerial part dry weight (DW) was determined by drying the plant tissues in a convection oven for 6 days at 105° C. (Provider: Memmert UN260). DW was measured using the same weighing scale than the one used to measure FW.

The quantification of relative chlorophyll concentration was performed through a non-destructive method that uses light transmission through a leaf, at two wavelengths, to determine the greenness and thickness of leaves. Transmission in the infrared range provides a measurement related to leaf thickness, and a wavelength in the red-light range is used to determine greenness. The ratio of the transmission of the two wavelengths provides a chlorophyll content index that is referred to as a Soil Plant Analysis Development (SPAD) index (Richardson et al., 2002).

A SPAD chlorophyll meter (SPAD-502 model, Minolta Camera Corporation Ltd., Osaka, Japan) was used to measure the SPAD index. The determination was carried out on 3 measurements per leaf from 6 plants per experimental condition. These measurements were done on the same foliar row for each plant (2nd leaf of middle thallus).

The number of biological replicates analyzed were 6 for plant growth (n=6) and 6 for SPAD (n=6). The observations are recorded below in Table 14.

TABLE 14
Efficacy of composition of Example 1 in wheat - fresh
biomass content, dry biomass content, SPAD index

| | Fresh | Dry | SPAD |
|---|---|---|---|
| Treatment | biomass (g) | biomass (g) | Index |
| Untreated | 12.27 | 1.5 | 32.9 |
| control | | | |
| Composition of | 16.13 | 1.89 | 39.78 |
| example 1 | | | |

It can be observed from example 5-9 that application of the compositions of the present disclosure led to an increase in growth parameters such as shoot weight, root weight, stem thickness, green index, chlorophyll content, fresh biomass, dry biomass and yield while delaying onset of senescence.

Examples 6-8 demonstrate that the compositions of the present disclosure can reduce dependency on fertilizer usage and provide better agricultural produce in nutrient deficient conditions.

1. A composition comprising a concentrated algal extract, a preservative and optionally, a diluent; wherein the concentrated algal extract is present in an amount ranging from about 50% w/w to about 99.75% w/w of the total weight of the composition, the concentration of the preservative is 0.15 w/w to 50% w/w of the total weight of the composition and the diluent is present in an in an amount ranging from about 0% w/w to about 50% w/w of the total weight of the composition.

2. The composition of claim 1, wherein the concentrated algal extract is characterized by presence of any one or more of biomarkers selected from the group consisting of
(a) Mannitol present in an amount ranging from about 1.5% w/w to about 7% w/w of the total weight of the extract;
(b) Fucoidans present in an amount ranging from about 0.4% w/w to about 1.5% w/w of the total weight of the extract;
(c) Polyphenols present in an amount ranging from about 0.2% w/w to about 5% w/w of the total weight of the extract;
(d) Glycine betaine present in an amount ranging from about 0.0001% w/w to about 0.005% w/w of the total weight of the extract; and
(e) Laminine present in an amount ranging from about 0.01% w/w to about 0.05% w/w of the total weight of the extract.

3. The composition of claim 1, wherein the concentrated algal extract is an acidic extract with a pH ranging between 2 and 4.

4. The composition of claim 1, wherein the concentrated algal extract is an extract of algae selected from Ascophyllum nodosum, Fucus vesiculosus, Laminaria digitata, Laminaria hyperborea, Laminaria saccharina, Ecklonia maxima, Sargassum spp., and their mixtures.

5. The composition of claim 1, wherein said composition comprises at least one preservative.

6. The composition of claim 1, wherein said composition comprises at least two preservatives.

7. The composition of claim 1, wherein the preservative is selected from 1,2-benzisothiazolin-3-one; 2-methyl-2H-isothiazol-3-one; sodium benzoate; potassium benzoate; benzoic acid; sorbic acid; potassium sorbate; sodium sulphite; potassium sulphite; sodium bisulphite; potassium bisulphite; sodium metabisulphite; potassium metabisulphite; sodium methylparaben; citric acid or salts thereof; malic acid or salts thereof; tartaric acid or salts thereof; propionic acid or salts thereof and combinations thereof.

8. The composition of claim 1, wherein said concentrated algal extract is prepared by a process comprising
i) crushing the algal cells to cause cell lysis;
ii) homogenizing the lysed algal cells in an aqueous solution;
iii) adding an acid to the homogenized lysed algal cells in an aqueous solution;
iv) removing alginate and cellulose by acidic flocculation and filtration; and
v) concentrating the filtrate to obtain the concentrated algal extract having a pH ranging between 2 and 4.

9. The composition of claim 8, wherein the pH of the filtrate is adjusted with a weak acid or mild acid selected from organic acids, oxalic acid, sulfurous acid, phosphoric acid, hydrogen sulfurous ion, nitrous acid, hydrofluoric acid, methanoic acid, benzoic acid, acetic acid, formic acid, citric acid, acetic acid, tartaric acid, ascorbic acid, lactic acid, uric acid, malic acid, succinic acid, propionic acid, acrylic acid, glyceric acid, carbonic acid, glycolic acid, glyoxylic acid, water, conjugates of weak bases, hydrocyanic and combinations thereof.

10. The composition of claim 8, wherein the extract obtained has a dry matter content of more than 5%.

11. The composition of claim 8, wherein the extract obtained has a total organic content of 5% to 100%.

12. A method of preparing the composition of claim 1, wherein said method comprises
(i) adding a preservative in an amount of about 0.15% w/w to about 50% w/w to the concentrated algal extract with dry matter more than 5% which is present in an amount of about 50% w/w to about 99.75% w/w, and
(ii) optionally adding a diluent to the composition obtained in step i) in an amount from about 0% w/w to about 50% w/w.

13. A method of growth stimulation in a target plant, wherein the method comprises applying to the target plant or a locus a composition as claimed in claim 1.

14. A method of determining the quality of the composition as claimed in claim 1, wherein said method comprises:
i) obtaining a sample of said composition; and
ii) measuring the pH and the concentration of any one or more of biomarkers selected from mannitol, glycine betaine, fucoidans, polyphenols and laminine in the sample of said composition;
wherein said composition is considered stable and good provided the pH of the sample of said composition is detected to be in a range of 2 to 4 and wherein the concentration of any one or more of biomarkers selected from mannitol, glycine betaine, fucoidans, polyphenols and laminine are detected to be in a range as provided in Table 2.
